(19) United States
(12) Patent Application Publication
(10) Pub. No.: US 2008/0014152 A1
(43) Pub. Date: Jan. 17, 2008

(54) INTRANASAL DELIVERY OF CLENBUTEROL ACROSS THE CRIBRIFORM PLATE AND INTO THE BRAIN

(76) Inventors: Thomas M. Di Mauro, Southboro, MA (US); Chantal Holy, Raynham, MA (US); Mohamed Attawia, Canton, MA (US); Sean Lilienfeld, Sharon, MA (US); Terri Kapur, Stoughton, PA (US); Francis Binette, Weysmouth, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/457,340

(22) Filed: Jul. 13, 2006

(51) Int. Cl. A61K 38/17; A61K 31/366; A61K 31/405; A61K 31/22; A61K 9/12; A61K 31/355

(52) U.S. Cl. 424/45; 514/419; 514/458; 514/460; 514/548; 514/12; 514/725

(57) ABSTRACT

The intranasal delivery of an effective amount of NGF inducers, such as clenbuterol, to the brain.

### BACKGROUND OF THE INVENTION

In Alzheimer's Disease (AD), the cleavage of beta amyloid protein precursor from the intracellular membrane often produces a protein AB-42 which is incompletely removed by normal clearance processes. It has been proposed that the soluble form of AB-42 is responsible for the local destruction of neurons. Over time, this protein is deposited as a beta amyloid protein Aβ plaque within brain tissue. The Aβ plaque deposition is also believed to provoke an inflammatory response by microglia and macrophages, which recognize the plaque as a foreign body. These cells are believed to respond to the plaque deposition by releasing pro-inflammatory cytokines and reactive oxygen species (ROS). Although the inflammatory response may be provoked in an effort to clear the brain tissue of the detrimental plaque, it is now believed that this inflammation also injures local neuronal tissue, thereby exacerbating AD.

Because of the role played in AD by inflammation, anti-inflammatory compounds have been identified as candidates for treating Alzheimer's Disease. However, the delivery of these compounds has generally been through an oral route, and the systemic side effects associated with long term use of these compounds are often undesirable.

### SUMMARY OF THE INVENTION

The present invention relates to the intranasal administration of NGF inducers such as clenbuterol across the cribriform plate and into the brain in order to treat neurogenerative diseases such as AD.

It has been suggested in the literature that orally active NGF inducers may have potential as therapeutic agents for the treatment of neurodegenerative disorders and stroke. Semkova, Brain Res. Brain Res. Rev., 1999 August 30(2) 176-88. However, the method by which Semkova suggested such inducers be administered is systemic (e.g., oral). It has been shown that chronic systemic administration of clenbuterol has problematic side effects, such as negative alteration of cardiac function. Sleeper, Med. Sci. Sports Exerc., 2002 April 34(4) 643-50. The trans-cribriform (local) method described herein provides an effective dosage of the NGF inducer to the target organ (the brain), but without the undesirable side effects related to its systemic administration.

In preferred embodiments, the NGF inducer is a β₂ adrenergic agonist, more preferably clenbuterol. Clenbuterol possesses a number of qualities that make attractive its intranasal administration across the cribriform plate and into the brain in order to treat neurodegenerative diseases such as AD.

First, two factors that will increase the intranasal delivery of a compound are high lipophilicity and low molecular weight. The high lipophilicity will allow it to be rapidly absorbed by the nasal mucosa and freely permeate across the olfactory mucosa (Kandimalla, Int. J. Pharm. 2005, Sep. 30, 302(1-2) 133-44). A low molecular weight will allow a compound to be easily transported across the blood brain barrier. Because clenbuterol is lipophilic (Semkova, Brain Res. Brain Res. Rev., 1999 August 30(2) 176-88), it should easily transport across the nasal mucosa and brain tissue. Moreover, since clenbuterol has a very low molecular weight, on the order of about 277 Daltons, it can be easily transported across the blood brain barrier.

Second, clenbuterol is a β₂ adrenergic agonist that induces an increase in the production of NGF in cortex neurons. Colangelo, PNAS USA 95, 1998, 10920-10925 reports that stimulation of β₂ adrenergic receptors by clenbuterol increases NGF biosynthesis in the rat cerebral cortex. Follesa, Mol. Pharmacol., 1993, February 43(2) 132-8 reports that a 10 mg/kg intraperitoneal dose of clenbuterol elicited a 2-3 fold increase in NGF mRNA in the cerebral cortex within 5 hours of administration.

The production of NGF within an AD brain is very important, as NGF is a neurotrophic factor essential to the development of cholinergic neurons in the basal forebrain, which play an important role in learning and memory processes. Colangelo, PNAS USA 95, 1998, 10920-10925. Of note, it also appears that there is an increase in β₂ adrenoreceptors in the prefrontal cortex in the AD patient. Kalaria, J. Neurochemistry, 53, 1772-81, 1989. Thus, since the intranasal delivery of a β₂ agonist such as clenbuterol across the cribriform plate is directed precisely towards the basal forebrain which has increased its β₂ adrenergic receptors, it is believed that intranasal delivery of a beta agonist is particularly well suited towards enhancing learning and memory processes in AD patients.

It is further believed that a significant portion of the clenbuterol will likely end up in the olfactory bulb, which resides just across the cribriform plate from the nasal cavity. Induction of NGF in the olfactory bulb is believed to be advantageous, since it has been reported that NGF is transported in a retrograde manner from the olfactory bulb forebrain cholinergic nuclei via the horizontal and vertical limbs of the diagonal band. Altar, Brain Res., 1991, Feb. 8, 541(1) 82-8.

Second, clenbuterol is a β₂ adrenergic agonist that induces the production of TGF-β by 2-3× in neurons. Zhu, Neuroscience, 2001, 107(4) 593-602, reports that a 0.5 mg/kg dose of clenbuterol increase TGF-β immunoreactivity as a early as 3 hours after administration, and remained elevated for up to 2 days thereafter. Zhu further reports that the administration of clenbuterol provided neuroprotection to hippocampal cells after ischemia.

Third, clenbuterol is a β₂ adrenergic agonist that induces the production of bFGF by 2-3× in neurons. Follesa Mol. Pharmacol., 1993, February 43(2) 132-8 reports that a 10 mg/kg intraperitoneal dose of clenbuterol elicited a 2-3 fold increase in bFGF mRNA in the cerebral cortex, hippocampus and cerebellum within 5 hours of administration.

Fourth, β₂ adrenergic agonists have been shown to promote anti-inflammatory responses. Izeboud, J. Recept. Signal Transduct. Res., 1999 January-July 19(1-4) 191-204 reports that the addition of clenbuterol to U937 cell stimulated with LPS acts to increase IL-10 production while decreasing TNF-α and IL-6 production. Abdulla, Biochem. Pharmacol., 2005 Mar. 1, 69(5) 741-50 reports that clenbuterol provides protection from inflammation in astrocytes treated with LPS.

Fifth, clenbuterol appears to selectively elevate NGF levels in the precise areas of the brain most affected by AD, such as the basal forebrain and the hippocampus.

For example, Zhu, J. Cereb. Blood Flow Metab., 1998 September 18(9) 1032-9 reports that in vivo stimulation of β₂ agonist receptors with intraperitoneal clenbuterol elevated NGF protein levels by 33% in the rat hippocampus. Semkova, Brain Res., 1996, Apr. 22, 717 (1-2) 44-54 reports that in vitro exposure of hippocampal cultures to clenbuterol enhanced significantly the NGF content in the culture medium, and concludes that the induction of NGF is mediated by β₂ adrenergic receptor activation. Of note, it also appears that there is an increase in β₂ adrenoreceptors in the hippocampus in the AD patient. Kalaria, J. Neurochemistry, 53, 1772-81, 1989.

Infusions of clenbuterol into the amygdala has been shown to enhance memory. McIntyre, PNAS USA 2005 Jul. 26, 102(30) 10718-23.

Therefore, in accordance with the present invention, there is provided a method of treating a neurodegenerative disease, comprising the steps of:

a) intranasally administering an effective amount of an NGF inducing agent such as clenbuterol across the cribriform plate and into the brain.

### DETAILED DESCRIPTION OF THE INVENTION

The 62 ₂-agonists of the present invention are known and can be obtained by the skilled person by conventional methods of chemical synthesis from readily available reagents. Many of these compounds are also commercially available from chemical suppliers (see the Merck Index). For example, clenbuterol may be obtained as described in U.S. Pat. No. 3,536,712 (incorporated herein by reference). Clenbuterol is also commercially available from Boehringer Ingelheim and Sigma Chemicals. Enantiomers of β₂-agonists such as clenbutarol may be obtained by methods known to the skilled chemist and are contemplated by this invention.

As discussed above, any compound having the activity of a β₂ agonist is useful in this invention. Particular β₂ agonists are albuterol, salmeterol, ractopamine, salbutamol, cimateril, BRL-47672, terbutaline, fenoterol, metaproterenol, isopraline, MJ-9184-1, trimetoquinol, tetrahydropapaveroline, soterenol, salmefamol, rimiterol, QH-25, isoetharine, R-804, ocripraline, quinterenol, sulfonterol, dobutamine, and isoproterenol. Preferred β₂ agonists are albuterol, salmeterol, ractopamine, salbutamol, cimateril, BRL-47672, terbutaline, fenoterol, metaproterenol, and isopraline. A particularly preferred β₂ agonist is clenbuterol. One or more β₂ agonists may be administered together, either simultaneously or at different times as part of the same treatment regimen. In this context, doses may be provided separately or combined in a single pharmaceutical composition.

It is further believed that another β₂ agonist, salmeterol, is also particularly suited for intranasal delivery across the cribriform plate and into the brain as a way of treating neurodegenerative disease. Lotvall, Respir. Med., 2001, August, 95 Suppl. B, S7-11, reports that salmeterol is highly lipophilic.

Specific dosage regimens for β₂ agonists in the method of this invention are from about 0.5 to about 1000.0 μg/kg/day. A range of from about 10.0 to about 100.0 μg/day is particularly effective, and about 40 μg/day is most effective. Thus for example clenbuterol may be administered in doses of from about 0.5 to about 1000.0 μg/kg/day, and in particular from about 10.0 to about 100.0 μg/day, preferably about 40 .mu.g/day. The word “about” in this context includes a range above and below the numbers provided, as would be considered reasonable by a skilled practitioner. If more than one β₂ agonist is administered in one dose, then the dosages of each should be adjusted (downward) accordingly.

Culmsee, Eur. J. Pharmacol., 1999, Aug. 20, 379(1) 33-45 reports that clenbuterol dosages greater than 1 mg/kg showed no cerebroprotective effect due to a decrease in blood pressure and an increase in plasma glucose level.

Pharmaceutical compositions containing β₂ agonists such as clenbuterol administered for the method of this invention are readily prepared by the skilled practitioner. Standard pharmaceutically acceptable inactive ingredients such as stabilizers, excipients, binding agents, carriers, vehicles, preservatives may be part of the compositions. More than one β₂ agonist may be used in a given composition. Other active ingredients may also be included.

The NGF inducing agent can be combined with a mucoadhesive to enhance its contact with the nasal mucosa. In some embodiments, the mucoadhesive is selected from the group consisting of a hydrophilic polymer, a hydrogel and a thermoplastic polymer. Preferred hydrophilic polymers include cellulose-based polymers (such as methylcellulose, hydroxyethyl cellulose, hydroxy propyl methyl cellulose, sodium carboxy methyl cellulose, a carbomer chitosan and plant gum.

In some embodiments, the mucoadhesive is a water-soluble high molecular weight cellulose polymer. High molecular weight cellulose polymer refers to a cellulose polymer having an average molecular weight of at least about 25,000, preferably at least about 65,000, and more preferably at least about 85,000. The exact molecular weight cellulose polymer used will generally depend upon the desired release profile. For example, polymers having an average molecular weight of about 25,000 are useful in a controlled-release composition having a time release period of up to about 8 hours, while polymers having an average molecular weight of about 85,000 are useful in a controlled-release composition having a time released period of up to about 18 hours. Even higher molecular weight cellulose polymers are contemplated for use in compositions having longer release periods. For example, polymers having an average molecular weight of 180,000 or higher are useful in a controlled-release composition having a time release period of 20 hours or longer.

The controlled-release carrier layer preferably consists of a water-soluble cellulose polymer, preferably a high molecular weight cellulose polymer, selected from the group consisting of hydroxypropyl methyl cellulose (HPMC), hydroxyethyl cellulose (HEC), hydroxypropyl cellulose (HPC), carboxy methyl cellulose (CMC), and mixtures thereof. Of these, the most preferred water-soluble cellulose polymer is HPMC. Preferably the HPMC is a high molecular weight HPMC, with the specific molecular weight selected to provide the desired release profile.

The HPMC is preferably a high molecular weight HPMC, having an average molecular weight of at least about 25,000, more preferably at least about 65,000 and most preferably at least about 85,000. The HPMC preferably consists of fine particulates having a particle size such that not less than 80% of the HPMC particles pass through an 80 mesh screen. The HPMC can be included in an amount of from about 4 to about 24 wt %, preferably from about 6 to about 16 wt % and more preferably from about 8 to about 12 wt %, based upon total weight of the composition.

Hydrogels can also be used to deliver the NGF inducer to the olfactory mucosa. A “hydrogel” is a substance formed when an organic polymer (natural or synthetic) is set or solidified to create a three-dimensional open-lattice structure that entraps molecules of water or other solution to form a gel. The solidification can occur, e.g., by aggregation, coagulation, hydrophobic interactions, or cross-linking. The hydrogels employed in this invention rapidly solidify to keep the NGF inducer at the application site, thereby eliminating undesired migration from the site. The hydrogels are also biocompatible, e.g., not toxic, to cells suspended in the hydrogel. A “hydrogel-inducer composition” is a suspension of a hydrogel containing desired NGF inducer. The hydrogel-inducer composition forms a uniform distribution of inducer with a well-defined and precisely controllable density. Moreover, the hydrogel can support very large densities of inducers. In addition, the hydrogel allows diffusion of nutrients and waste products to, and away from, the inducer, which promotes tissue growth.

Hydrogels suitable for use in the present invention include water-containing gels, i.e., polymers characterized by hydrophilicity and insolubility in water. See, for instance, “Hydrogels”, pages 458-459 in Concise Encyclopedia of Polymer Science and Engineering, Eds. Mark et al., Wiley and Sons, 1990, the disclosure of which is incorporated herein by reference.

In a preferred embodiment, the hydrogel is a fine, powdery synthetic hydrogel. Suitable hydrogels exhibit an optimal combination of such properties as compatibility with the matrix polymer of choice, and biocompatibility. The hydrogel can include any of the following: polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers. Other preferred hydrogels include poly(acrylic acid co acrylamide) copolymer, carrageenan, sodium alginate, guar gum and modified guar gum.

In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

Preferred thermoplastic polymers include PVA, polyamide, polycarbonate, polyalkylene glycol, polyvinyl ether, polyvinyl ether, and polyvinyl halides, polymethacrylic acid, polymethylmethacrylic acid, methyl cellulose, hydroxypropyl cellulose, hydroxypropyl methylcellulose, and sodium carboxymethylcellulose, ethylene glycol copolymers,

Other polymers that may be suitable for use as a mucoadhesive include aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as collagen, elastin, bioabsorbable starches, etc.) and blends thereof. For the purpose of this invention aliphatic polyesters include, but are not limited to, homopolymers and copolymers of lactide (which includes lactic acid, D-,L- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, χ-butyrolactone, ε-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one, 2,5-diketomorpholine, pivalolactone, χ,χ-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof. Poly(iminocarbonates), for the purpose of this invention, are understood to include those polymers as described by Kemnitzer and Kohn, in the Handbook of Biodegradable Polymers, edited by Domb, et. al., Hardwood Academic Press, pp. 251-272 (1997). Copoly(ether-esters), for the purpose of this invention, are understood to include those copolyester-ethers as described in the Journal of Biomaterials Research, Vol. 22, pages 993-1009, 1988 by Cohn and Younes, and in Polymer Preprints (ACS Division of Polymer Chemistry), Vol. 30(1), page 498, 1989 by Cohn (e.g. PEO/PLA). Polyalkylene oxalates, for the purpose of this invention, include those described in U.S. Pat. Nos. 4,208,511; 4,141,087; 4,130,639; 4,140,678; 4,105,034; and 4,205,399. Polyphosphazenes, co-, ter- and higher order mixed monomer-based polymers made from L-lactide, D,L-lactide, lactic acid, glycolide, glycolic acid, para-dioxanone, trimethylene carbonate and ε-caprolactone such as are described by Allcock in The Encyclopedia of Polymer Science, Vol. 13, pages 31-41, Wiley Intersciences, John Wiley & Sons, 1988 and by Vandorpe, et al in the Handbook of Biodegradable Polymers, edited by Domb, et al, Hardwood Academic Press, pp. 161-182 (1997). Polyanhydrides include those derived from diacids of the form HOOC—C₆H₄—O—(CH₂)ₘ—O—C₆H₄—COOH, where m is an integer in the range of from 2 to 8, and copolymers thereof with aliphatic alpha-omega diacids of up to 12 carbons. Polyoxaesters, polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213; 5,700,583; and 5,859,150. Polyorthoesters such as those described by Heller in Handbook of Biodegradable Polymers, edited by Domb, et al, Hardwood Academic Press, pp. 99-118 (1997).

In some embodiments, the mucoadhseive is selected from the group consisting of poly(lactic acid) (“PLA”) and poly(glycolic acid)(“PGA”), and copolymers thereof.

In some embodiments, the mucoadhesive formulation includes a penetration enhancer such as sodium glycocholate, sodium taurocholate, L-lysophosphotidyl choline, DMSO and a protease inhibitor.

In some embodiments, the beta agonist is tagged with a molecule that binds specifically with the olfactory mucosa.

In some embodiments, the beta agonist is combined with a second lipophilic therapeutic agents. In some embodiments thereof, the second lipophilic therapeutic agent is selected from the group consisting of Vitamin A, Vitamin E, melatonin, lovastatin, and VIP analog. Vitamin A is an anti-oxidant. Vitamin E is an anti-oxidant. Melatonin is an anti-oxidant. Lovastatin decreases BAP production. VIP enhances cholinergic function.

Preferably, the intranasal procedure of the present invention can be applied to patients suffering from Alzheimer's Disease or stroke.

In some embodiments, the intranasal administration of the beta agonist across the cribriform plate is used to treat a stroke patient. Culmsee, Stroke, 2004:35:1197-1202 reports that clenbuterol has been demonstrated to show neuroprotective capacity in experimental models of stroke (citing 4 references). Zhu, J. Cereb. Blood Flow Metab., 1998 September 18(9) 1032-9 reports that stimulation of β₂ agonist receptors inhibited apoptosis in the rat brain after transient forebrain ischemia. Rami, Neurosci. Res., 2003 December 47(4), 373-82 reports that clenbuterol attenuates apoptosis in the rat hippocampus after transient global ischemia. Culmsee, Eur. J. Pharmacol., 1999, Aug. 20, 379(1) 33-45 reports that 0.01-0.5 mg/kg clenbuterol reduced cortical infarct volume in rats as measured 7 days after permanent occlusion of the middle cerebral artery.

In some embodiments, the intranasal administration of the beta agonist across the cribriform plate is used to treat a patient with amyotrophic lateral sclerosis (ALS).

For delivery, there is provided a standard nose drops squeezable spray container with a long thin semi-flexible tube attached to the distal end. The outer diameter of the tube is less than a millimeter, preferably less than 0.5 mm, more preferably less than 0.25 mm. The exit hole of the tube is preferably located on the peripheral wall near the distal end of the tube so that spray exiting it can be directed upwards. There is a marker on the container that indicates when the exit hole is oriented upwards towards the cribriform plate.

Therefore, in accordance with the present invention, there is provided an intranasal spray device comprising:
a) a hollow container having a first opening,
b) a flexible tube having a throughbore, a side surface having a second opening, a proximal end having a third opening, and a distal end having an end surface,
c) a formulation comprising a CNS therapeutic agent contained within the container,
   wherein the third opening of the proximal end of the tube is in fluid connection with the first opening of the hollow container.

The user directs the tube towards the medial wall of the nostril and points upwards so as to direct it medial to and over the middle nasal concha. The length of the tube is predetermined so that when the user has the shoulder of the container flush against the nostril the hole is adjacent the cribriform plate.

If there is concern about the safety of inserting a tube through a nasal passage, then the tube can also be balloon-like, so that it expands to full length upon being pressurized.

In some embodiments, the beta agonist is delivered iontophoretically. Preferably, the beta agonist delivered iontophoretically is clenbuterol. Jones, Brain Res., 1986, Mar. 5, 367 (1-2) 151-61 reports that the iontophoresis of clenbuterol with low currents on the order of 15 nA. In some embodiments, the iontophoresis is carried out in accordance with U.S. Ser. No. 11/200,438, entitled “Methods of Delivering Therapeutics to the Brain”, filed Aug. 8, 2005, (COD-5112), the specification of which is incorporated by reference in its entirety.

In some embodiments, the NGF inducer is combined with microparticles and the delivery of the microparticle is assisted by applying ultrasound thereto.

In some embodiments, the NGF inducer is combined with diamagnetic pyrolytic graphite microparticles and the delivery of the microparticle to the olfactory mucosa is assisted by assisted by applying a magnetic field to the pyrolytic graphite particles.

In some embodiments, the NGF inducing agent (preferably a beta agonist) is administered intrathecally through a drug pump, preferably through a lumbar puncture.

We claim:

1. A method of treating a neurodegenerative disease, comprising the steps of:
a) intranasally administering an effective amount of an NGF inducing agent across the cribriform plate and into the brain.

2. The method of claim 1 wherein the NGF inducing agent is a β₂ adrenergic agonist

3. The method of claim 2 wherein the β₂ adrenergic agonist is clenbuterol.

4. The method of claim 2 wherein the clenbuterol is administered in an amount of between about 10.0 μg/day to about 1000.0 μg/day.

5. The method of claim 2 wherein the beta agonist is administered in a formulation.

6. The method of claim 5 wherein the beta agonist is tagged with a molecule that binds specifically with the olfactory mucosa

7. The method of claim 6 wherein the formulation further comprises a second lipophilic therapeutic agents.

8. The method of claim 7 wherein the second lipophilic therapeutic agent is selected from the group consisting of Vitamin A, Vitamin E, melatonin, lovastatin, and VIP analog.

9. The method of claim 5 wherein the formulation further comprises a mucoadhesive.

10. The method of claim 5 wherein the formulation further comprises a penetration enhancer selected from the group consisting of sodium glycocholate, sodium taurocholate, L-lysophosphotidyl choline, DMSO and a protease inhibitor.

11. An intranasal spray device comprising:
a) a hollow container having a first opening,
b) a flexible tube having a throughbore, a side surface having a second opening, a proximal end having a third opening, and a distal end having an end surface,
c) a formulation comprising a CNS therapeutic agent contained within the container,
wherein the third opening of the proximal end of the tube is in fluid connection with the first opening of the hollow container.

12. The device of claim 11 wherein the CNS therapeutic agent is an NGF inducing agent.

13. The device of claim 12 wherein the MGF inducing agent is a beta adrenergic agonist.

14. The device of claim 12 wherein the beta adrenergic agonist is clenbuterol.

15. The device of claim 11 wherein the hollow container is flexible.

16. The device of claim 11 wherein the hollow container has a height, and wherein the flexible tube has a length, wherein the length of the flexible tube is greater than the height of the hollow container.

17. A formulation for intranasal delivery to the brain, comprising:
a) a lipophilic NGF inducing agent, and
b) a second lipophilic therapeutic agent.

18. The formulation of claim 17 wherein the lipophilic NGF inducing agent is a beta adrenergic agonist.

19. The formulation of claim 17 wherein the beta adrenergic agonist is selected from the group consisting of clenbuterol and salmeterol.

20. The formulation of claim 17 wherein the second lipophilic therapeutic agent is selected from the group consisting of Vitamin A, Vitamin E, melatonin, lovastatin, and VIP analog.

21. A method of treating a neurodegenerative disease, comprising the steps of:
a) providing an intranasal spray device comprising:
i) a hollow container having a first opening,
ii) a flexible tube having a throughbore, a side surface, a proximal end having a second opening, and a distal end portion having a third opening, and
iii) a formulation comprising a NGF inducing agent contained within the container,
wherein the third opening of the proximal end of the tube is in fluid connection with the first opening of the hollow container, and
b) inserting the distal end of the flexible tube into a nostril,
c) advancing the third opening of the distal end portion of the flexible tube to a location adjacent the olfactory mucosa, and
d) moving the formulation from the device through the flexible tube to the olfactory mucosa.

22. The method of claim 21 wherein the NGF inducing agent is a beta adrenergic agonist.

23. The method of claim 22 wherein the β₂ adrenergic agonist is clenbuterol.

24. The method of claim 22 wherein the clenbuterol is administered in an amount of between about 10.0 μg/day to about 100.0 μg/day.

25. The method of claim 22 wherein the beta agonist is administered in a formulation.

26. The method of claim 25 wherein the beta agonist is lipophilic.

27. The method of claim 26 wherein the formulation further comprises a second lipophilic therapeutic agents.

28. The method of claim 27 wherein the second lipophilic therapeutic agent is selected from the group consisting of Vitamin A, Vitamin E, melatonin, lovastatin, and VIP analog.

29. The method of claim 25 wherein the formulation further comprises a mucoadhesive.

30. The method of claim 25 wherein the formulation further comprises a penetration enhancer.

31. A method of treating a neurodegenerative disease, comprising the steps of:
a) intrathecally administering an effective amount of an NGF inducing agent into the brain.

32. The method of claim 31 wherein the NGF inducing agent is a beta agonist.

33. The method of claim 32 wherein the beta agonist is intrathecally administered via a drug pump.

34. The method of claim 32 wherein the beta agonist is intrathecally administered via a lumbar puncture.
